(19) United States
(12) Patent Application Publication
(10) Pub. No.: US 2023/0021127 A1
(43) Pub. Date: Jan. 19, 2023

(54) STABLE COMPOSITION COMPRISING COMBINATION OF CINNAMIC ACID DERIVATIVES WITH SPECIFIC INGREDIENTS

(71) Applicant: L'OREAL, PARIS (FR)

(72) Inventor: Kazuhiko MARUYAMA, Kawasaki-shi, Kanagawa (JP)

(21) Appl. No.: 17/757,207

(22) PCT Filed: Dec. 4, 2020
(86) PCT No.: PCT/JP2020/046189

§ 371 Date: Jun. 10, 2022

(30) Foreign Application Priority Data
Dec. 16, 2019 (JP) 2019-226592

(51) Int. Cl. A61K 8/365; A61K 8/11; A61K 8/37; A61K 8/42; A61K 8/67; A61K 8/73; A61K 8/92

(52) U.S. Cl. CPC A61K 8/365; A61K 8/11; A61K 8/37; A61K 8/42; A61K 8/678; A61K 8/73; A61K 8/92; A61K 2800/522

(57) ABSTRACT

The present invention relates to a composition comprising: (a) at least one cinnamic acid derivative according to a specific chemical formula; (b) at least one lipophilic antioxidant; (c) at least one oil; and (d) water, wherein the pH of the composition is 4.5 or less. The composition according to the present invention is stable under a variety of temperature conditions.

### TECHNICAL FIELD

The present invention relates to a composition, preferably a cosmetic or dermatological composition, which comprises a combination of specific ingredients.

### BACKGROUND ART

Cinnamic acid and derivatives thereof, such as ferulic acid, have been known to be useful in the field of, for example, cosmetics because they can function as a UV filter, an antioxidant and the like. However, they tend to be unstable under some conditions. For example, ferulic acid is soluble in water under alkaline conditions, but the aqueous solution of ferulic acid may rapidly discolor (yellowing) and give off a bad smell.

Cinnamic acid and derivatives thereof also have a problem in that they tend to become discolored, crystalize and give off a bad smell in cosmetic compositions due to not only pH but also temperature (storage) conditions. However, sufficient investigations have not as yet been made for the technique of effectively preventing the discoloration, the crystallization and the bad smell of a composition including cinnamic acid and derivatives.

### DISCLOSURE OF INVENTION

An objective of the present invention is to provide a composition which is stable under a variety of temperature conditions, even if the composition includes a cinnamic acid derivative, such as ferulic acid, and water.

The above objective of the present invention can be achieved by a composition comprising:

(a) at least one cinnamic acid derivative represented by the following chemical formula (I)

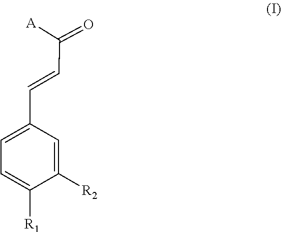

wherein
A is chosen from
   an OR₃ group wherein R₃ is chosen from a hydrogen atom, a phytyl group, a benzyl group, a linear or branched C₁-C₁₈ alkyl group, a C₃-C₈ cycloalkyl group, a C₃-C₈ cycloalkyl-C₁-C₅ alkyl group, an alkali metal ion, an alkaline earth metal ion and an ammonium ion,
and
   an NHR₄ group wherein R₄ is chosen from a hydrogen atom, a phytyl group, a benzyl group, and a linear or branched C₁-C₁₈ alkyl group, a C₃-C₈ cycloalkyl group, a C₃-C₈ cycloalkyl-C₁-C₅ alkyl group,
R₁ is chosen from a hydrogen atom, a hydroxyl group, a C₁-C₆ alkoxy group, a linear or branched C₁-C₁₈ alkyl group, a C₃-C₈ cycloalkyl group, a C₃-C₈ cycloalkyl-C₁-C₅ alkyl group, and
R₂ is chosen from a hydrogen atom, a hydroxyl group, and a C₁-C₆ alkoxy group;
   (b) at least one lipophilic antioxidant;
   (c) at least one oil; and
   (d) water,
   wherein
   the pH of the composition is 4.5 or less.

The (a) cinnamic acid derivative may be ferulic acid.

The amount of the (a) cinnamic acid derivative(s) in the composition according to the present invention may range from 0.01% to 8% by weight, preferably from 0.05% to 4% by weight, and more preferably from 0.1% to 2% by weight, relative to the total weight of the composition.

The (b) lipophilic antioxidant may be selected from tocopherol, pentaerythrityl tetra-di-t-butyl hydroxyhydrocinnamate, and a mixture thereof.

The amount of the (b) lipophilic antioxidant(s) in the composition according to the present invention may range from 0.001% to 10% by weight, preferably from 0.005% to 5% by weight, and more preferably from 0.01% to 2% by weight, relative to the total weight of the composition.

It is preferable that the (a) cinnamic acid derivative be soluble in the (c) oil.

The (c) oil may be selected from the group consisting of isopropyl lauroyl sarcosinate, bis-ethoxydiglycol cyclohexane 1,4-dicarboxylate, and mixtures thereof.

The amount of the (c) oil(s) in the composition according to the present invention may range from 0.01% to 50% by weight, preferably from 0.1% to 30% by weight, and more preferably from 1% to 15% by weight, relative to the total weight of the composition.

The amount of the (d) water in the composition according to the present invention may range from 50% to 90% by weight, preferably from 60% to 85% by weight, and more preferably from 70% to 80% by weight, relative to the total weight of the composition.

The composition according to the present invention may further comprise (e) at least one polysaccharide, preferably selected from polysaccharides derived from plants, and more preferably selected from algal extracts.

The (e) polysaccharide may be selected from agar, alginate, carrageenan and a mixture thereof.

The (e) polysaccharide(s) in the composition according to the present invention may range from 0.001% to 5% by weight, preferably from 0.005% to 2% by weight, and more preferably from 0.01% to 1% by weight, relative to the total weight of the composition.

The composition according to the present invention may be is in the O/W form, preferably an O/W emulsion or an O/W dispersion, and more preferably an O/W gel emulsion or an O/W gel dispersion.

The composition according to the present invention may be a cosmetic composition, preferably a cosmetic composition for a keratin substance, and more preferably a cosmetic composition for skin.

The present invention also relates to a cosmetic process for treating a keratin substance, comprising the step of applying the composition according to the present invention to the keratin substance.

### BEST MODE FOR CARRYING OUT THE INVENTION

After diligent research, the inventors have discovered that it is possible to provide a composition which is stable under a variety of temperature conditions, even if the composition includes a cinnamic acid derivative, such as ferulic acid, and water.

The composition according to the present invention may be characterized by a combination of:

at least one cinnamic acid derivative according to the above chemical formula (I);
at least one lipophilic antioxidant;
at least one oil; and
water
under a pH of 4.5 or less.

Thus, one aspect of the present invention is a composition comprising:

(a) at least one cinnamic acid derivative according to the above chemical formula (I);
(b) at least one lipophilic antioxidant; and
(c) at least one oil;
(d) water,
wherein
the pH of the composition is 4.5 or less.

The composition according to the present invention is stable under a variety of temperature conditions. Thus, the term “stable” here reflects a temperature stability and means that the aspect of a composition does not change for a long period of time at a variety of constant temperatures such as 4° C., 25° C., 40° C. and 45° C.

Since the composition according to the present invention is stable, it can cause, for example, no or little discoloration, such as yellowing, crystallization, and bad odor, for a long period of time.

The composition according to the present invention can be stored for a long period of time, because it can cause no or reduced changes such as yellowing, crystallization, and bad odor.

Accordingly, the composition according to the present invention can provide stable cosmetic effects over time based on the cinnamic acid derivative in the composition.

Hereinafter, the composition according to the present invention will be explained in a more detailed manner.

[Cinnamic Acid Derivative]

The composition according to the present invention comprises (a) at least one cinnamic acid derivative with a specific chemical structure. Two or more the (a) cinnamic acid derivatives may be used in combination. Thus, a single type of the cinnamic acid derivative or a combination of different types of cinnamic acid derivative may be used.

The (a) cinnamic acid derivative is represented by the following chemical formula (I).

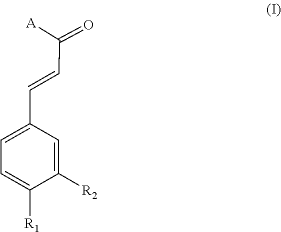

wherein
A is chosen from
an OR₃ group wherein R₃ is chosen from a hydrogen atom, a phytyl group, a benzyl group, a linear or branched C₁-C₁₈ alkyl group, a C₃-C₈ cycloalkyl group, a C₃-C₈ cycloalkyl-C₁-C₅ alkyl group, an alkali metal ion, an alkaline earth metal ion and an ammonium ion,
   and
an NHR₄ group wherein R₄ is chosen from a hydrogen atom, a phytyl group, a benzyl group, and a linear or branched C₁-C₁₈ alkyl group, a C₃-C₈ cycloalkyl group, a C₃-C₈ cycloalkyl-C₁-C₅ alkyl group,
   R₁ is chosen from a hydrogen atom, a hydroxyl group, a C₁-C₆ alkoxy group, a linear or branched C₁-C₁₈ alkyl group, a C₃-C₈ cycloalkyl group, a C₃-C₈ cycloalkyl-C₁-C₅ alkyl group, and
   R₂ is chosen from a hydrogen atom, a hydroxyl group, and a C₁-C₆ alkoxy group;

As the linear or branched C₁-C₁₈ alkyl group, preferably C₁-C₁₂ alkyl group, and more preferably C₁-C₆ alkyl group, mention may be made of, for example, a methyl group, an ethyl group, an n-propyl group, an i-propyl group, an n-butyl group, an i-butyl group, a sec-butyl group, a tert-butyl group, an n-pentyl group, an i-pentyl group, a 1-ethylpropyl group, a hexyl group, an isohexyl group, and a 1-ethylbutyl group.

As the C₃-C₈ cycloalkyl group, mention may be made of, for example, a cyclopropyl group, a cyclobutyl group, a cyclopentyl group, and a cyclohexyl group.

As the C₃-C₈ cycloalkyl-C₁-C₅ alkyl group, mention may be made of, for example, a cyclopropylmethyl group, a cyclobutylmethyl group, a cyclopentylmethyl group, and a cyclohexylmethyl group.

As the C₁-C₆ alkoxy group, mention may be made of, for example, a methoxy group, an ethoxy group, an n-propoxy group, an i-propoxy group, an n-butoxy group, an i-butoxy group, a sec-butoxy group, a tert-butoxy group, an n-pentyoxy group, an i-pentyoxy group, a 1-ethylpropoxy group, a hexyloxy group, an isohexyloxy group, and a 1-ethylbutoxy group. A methoxy group is preferable.

It may be preferable that R₁ be a hydroxyl group, and that R₂ be chosen from a hydroxyl group and a C₁-C₆ alkoxy group, more preferably a methoxy group.

As the (a) cinnamic acid derivative, mention may be made of, for example, 2-ethylhexyl methoxycinnamate, isopropyl methoxycinnamate, isoamyl methoxycinnamate, diisopropyl methoxycinnamate, caffeic acid, ferulic acid. Caffeic acid and ferulic acid may be preferable, and ferulic acid may be more preferable.

The (a) cinnamic acid derivative may be an active ingredient or active compound in cosmetics or dermatological products. The term an “active” compound used herein means a compound which has a cosmetic or dermatological active property, such as anti-oxidant, whitening, and UV-filtering effects. Preferably, the (a) cinnamic acid derivative used in the present invention may be a UV filter, and thus the composition according to the present invention may be used as a UV-protecting products or as a cosmetic composition for protecting keratin substance such as skin from UV rays.

The amount of the (a) cinnamic acid derivative(s) in the composition according to the present invention may be 0.01% by weight or more, preferably 0.05% by weight or more, and more preferably 0.1% by weight or more, relative to the total weight of the composition. It may be even more preferable that the amount of the (a) cinnamic acid derivative(s) in the composition according to the present invention be 0.2% by weight or more, relative to the total weight of the composition.

On the other hand, the amount of the (a) cinnamic acid derivative(s) in the composition according to the present invention may be 8% by weight or less, preferably 4% by weight or less, and more preferably 2% by weight or less, relative to the total weight of the composition. It may be even more preferable that the amount of the (a) cinnamic acid derivative(s) in the composition according to the present invention be 1% by weight or less, relative to the total weight of the composition.

The amount of the (a) cinnamic acid derivative(s) in the composition according to the present invention may range from 0.01% to 8% by weight, preferably from 0.05% to 4% by weight, more preferably from 0.1% to 2% by weight, relative to the total weight of the composition. It may be even more preferable that the amount of the (a) cinnamic acid derivative(s) in the composition according to the present invention be from 0.2% to 1% by weight, relative to the total weight of the composition.

[Lipophilic Antioxidant]

The composition according to the present invention comprises (b) at least one lipophilic antioxidant agent. A single type of lipophilic antioxidant agent may be used, but two or more different types of lipophilic antioxidant agent may be used in combination.

According to the present invention, antioxidant agents are compounds or substances that can scavenge the various radical forms which may be present in the skin; preferably, they simultaneously scavenge all the various radical forms present.

The (b) lipophilic antioxidant is different from the (a) cinnamic acid derivative according to the above chemical formula (I).

As antioxidant agents, mention may be made of, phenolic antioxidants which have a hindered phenol structure or a semi-hindered phenol structure within the molecule. As specific examples of such compounds, mention may be made of 3,5-bis(1,1-dimethylethyl)-4-hydroxybenzenepropanoic acid) which has the INCI name of pentaerythrityl tetra-di-t-butyl hydroxyhydrocinnamate, 2,6-di-tert-butyl-4-methylphenol, 2,6-di-tert-butyl-4-ethylphenol, mono- or di- or tri-(α-methylbenzyl)phenol, 2,2′-methylenebis(4-ethyl-6-tert-butylphenol), 2,2′-methylenebis(4-methyl-6-tert-butylphenol), 4,4′-butylidenebis(3-methyl-6-tert-butylphenol), 4,4′-thiobis(3-methyl-6-tert-butylphenol), 2,5-di-tert-butylhydroquinone, 2,5-di-tert-amylhydroquinone, tris[N-(3,5-di-tert-butyl-4-hydroxybenzyl)]isocyanurate, 1,1,3-tris(2-methyl-4-hydroxy-5-tert-butylphenyl)butane, butylidene-1,1bis[3-(3-tert-butyl-4-hydroxy-5-methylphenyl)propionate], octadecyl 3-(3,5-di-tert-butyl-4-hydroxyphenyl)propionate, tetrakis[methylene-3-(3,5-di-tert-butyl-4-hydroxyphenyl)propionato]methane, triethylene glycol bis[3-(3-tert-butyl-4-hydroxy-5-methylphenyl)propionate], 3,9-bis{2-[3-(3-tert-butyl-4-hydroxy-5-methylphenyl)propionyloxy]-1,1-dimethylethyl}-2,4,8,10-tetraoxaspiro [5.5]undecane, 1,3,5-trimethyl-2,4,6-tris(3,5-di-tert-butyl-4-hydroxybenzyl)benzene, 2,2-thiodiethylenebis[3-(3,5-di-tert-butyl-4-hydroxyphenyl)propionate], N,N′-hexamethylenebis(3,5-di-tert-butyl-4-hydroxyhydrocinnamide), 1,6-hexanediol bis[3-(3,5-di-tert-butyl-4-hydroxyphenyl)propionate], 1,3,5-tris[(4-tert-butyl-3-hydroxy-2,6-xylyl)methyl]-1,3,5-triazine-2,4,6-trione, 2,4-bis(n-octylthio)-6-(4-hydroxy-3,5-di-tert-butylanilino)-1,3,5-triazine, 2-tert-butyl-6-(3′-tert-butyl-5′-methyl-2′-hydroxybenzyl)-4-methylphenyl acrylate, 2-[l-(2-hydroxy-3,5-di-tert-pentylphenyl)ethyl]-4,6-di-tert-pentylphenyl acrylate, 4,6-bis[(octylthio)methyl]-o-cresol, 2,4-di-tert-butylphenyl-3,5-di-tert-butyl-4-hydroxybenzoate and 1,6-hexanediolbis[3-(3,5-di-tert-butyl-4-hydroxyphenyl) propionate].

As antioxidant agents, mention may be made of: BHA (butylated hydroxyl anisole) and BHT (butylated hydroxyl toluene), vitamin E (or tocopherols and tocotrienol) and derivatives thereof, such as the phosphate derivative, for instance TPNA® sold by the company Showa Denko, coenzyme Q10 (or ubiquinone), idebenone, certain carotenoids such as lutein, astaxanthin, beta-carotene, polyphenols, phenolic acids and derivatives (e. g., chlorogenic acid), and flavonoids, which represent the main subgroup of polyphenols.

Among the flavonoids, mention may be made especially of chalcones, hydroxylated chalcones and reduced derivatives thereof (as described especially in patent FR 2 608 150), for instance phloretin, neohesperidin, phloridzin, aspalathin, etc., flavanones, for instance hesperetin and naringin, flavonols, for instance quercetin, rutin, flavanols, for instance catechin, EGCG, flavones, for instance apigenidin, and finally anthocyans. Mention may also be made of tannins. Reference may also be made to the compounds described in patent applications FR 2 699 818, FR 2 706 478, FR 2 907 339, FR 2 814 943 and FR 2 873 026.

Polyphenol compounds may especially be derived from plant extracts chosen from extracts of green tea, apple, hop, guava, cocoa or wood, such as chestnut, oak, horse chestnut or hazel. It is also possible to use an extract of pinaster bark, for example obtained according to processes described in U.S. Pat. Nos. 4,698,360, 6,372,266 and 5,720,956. As examples of such extracts, the compound referenced as INCI name Pinus pinaster (bark extract) and as CTFA name Pine (Pinus pinaster) bark extract may be cited. It may in particular be the extract of pinaster bark marketed under the PYCNOGENOL® reference by the BIOLANDES AROMES firm and/or HORPHAG Research. The extracts (Maritime) pine bark from LAYN Natural Ingredients, Pine Bark from Blue California, and also Oligopin® from D.R.T. (Les Derives Resiniques et Terpeniques) may also be cited.

In the context of the present invention, the term “polyphenol compound” thus also covers the plant extract itself, rich in these polyphenol compounds.

Antioxidants that may also be mentioned include dithiolanes, for instance asparagusic acid, or derivatives thereof, for instance siliceous dithiolane derivatives, especially such as those described in patent application FR 2 908 769.

Antioxidants that may also be mentioned include:

glutathione and derivatives thereof (GSH and/or GSHOEt), such as glutathione alkyl esters (such as those described in patent applications FR 2 704 754 and FR 2 908 769);
cysteine and derivatives thereof, such as N-acetylcysteine or L-2-oxothiazolidine-4-carboxylic acid. Reference may also be made to the cysteine derivatives described in patent applications FR 2 877 004 and FR 2 854 160;
certain enzymes for defending against oxidative stress, such as catalase, superoxide dismutase (SOD), lactoperoxidase, glutathione peroxidase and quinone reductases;
benzylcyclanones; substituted naphthalenones; pidolates (as described especially in patent application EP 0 511 118); caffeic acid and derivatives thereof, gamma-oryzanol; melatonin, sulforaphane and extracts containing it (excluding cress);
the diisopropyl ester of N,N′-bis(benzyl)ethylenediamine-N,N′-diacetic acid, as described especially in patent applications WO 94/11338, FR 2 698 095, FR 2 737 205 or EP 0 755 925; deferoxamine (or desferal) as described in patent application FR 2 825 920.

The antioxidants that are preferably used are the chalcones, more particularly phloretin or neohesperidin, the diisopropyl ester of N,N′-bis(benzyl)ethylenediamine-N,N′-diacetic acid or an extract of pinaster bark such as PYCNOGENOL®.

The (b) lipophilic antioxidant agent means that the partition coefficient of the antioxidant agent between n-butanol and water is >1, more preferably >10 and even more preferably >100.

As examples of the (b) lipophilic antioxidant agent, mention may be made of pentaerythrityl tetra-di-t-butyl hydroxyhydrocinnamate, nordihydroguaiaretic acid, tocopherol, resveratrol, propyl gallate, butylated hydroxyl toluene, butylated hydroxyl anisole, ascorbyl palmitate, tocopherol, and mixtures thereof.

It is preferable that the (b) lipophilic antioxidant be selected from tocopherol, pentaerythrityl tetra-di-t-butyl hydroxyhydrocinnamate, and a mixture thereof.

The amount of the (b) lipophilic antioxidant(s) in the composition according to the present invention may be 0.001% by weight or more, preferably 0.005% by weight or more, and more preferably 0.01% by weight or more, relative to the total weight of the composition. It may be even more preferable that the amount of the (b) lipophilic antioxidant(s) in the composition according to the present invention be 0.02% by weight or more, relative to the total weight of the composition.

On the other hand, the amount of the (b) lipophilic antioxidant(s) in the composition according to the present invention may be 10% by weight or less, preferably 5% by weight or less, and more preferably 2% by weight or less, relative to the total weight of the composition. It may be even more preferable that the amount of the (b) lipophilic antioxidant(s) in the composition according to the present invention be 1% by weight or less, relative to the total weight of the composition.

The amount of the (b) lipophilic antioxidant(s) in the composition according to the present invention may range from 0.001% to 10% by weight, preferably from 0.005% to 5% by weight, and more preferably from 0.01% to 2% by weight, relative to the total weight of the composition. It may be even more preferable that the amount of the (b) lipophilic antioxidant(s) in the composition according to the present invention be from 0.02% to 1% by weight, relative to the total weight of the composition.

[Oil]

The composition according to the present invention comprises (c) at least one oil. Two or more (c) oils may be used in combination. Thus, a single type of oil or a combination of different types of oils may be used.

Here, “oil” means a fatty compound or substance which is in the form of a liquid or a paste (non-solid) at room temperature (25° C.) under atmospheric pressure (760 mmHg). As oils, those generally used in cosmetics can be used alone or in combination thereof. These oils may be volatile or non-volatile.

It is preferable that the (a) cinnamic acid derivative according to the above chemical formula (I) be soluble in the (c) oil. In other words, it is preferable to use the (c) oil in which the (a) cinnamic acid derivative according to the above chemical formula (I) is soluble.

It is preferable that the solubility of the (a) cinnamic acid derivative to the (c) oil be 0.5% or more, more preferably 0.7% or more, and even more preferably 0.9% or more, at room temperature (20-25° C., preferably 25° C.). It may be preferable that the solubility of the (a) cinnamic acid derivative to the (c) oil be 25% or less, more preferably 20% or less, and even more preferably 15% or less, at room temperature (20-25° C., preferably 25° C.). Thus, the solubility of the (a) cinnamic acid derivative to the (c) oil may be from 0.5% to 25%, preferably from 0.7% to 20%, and more preferably from 0.9% to 15%. The solubility here means an amount (g) of the (a) cinnamic acid derivative which is soluble in 100 g of the (c) oil.

It is preferable that the (c) oil be selected from polar oils.

The term “polar oil” here means any lipophilic compound having, at 25° C., a solubility parameter δ_{d} characteristic of dispersive interactions of greater than 16 and a solubility parameter δₚ characteristic of polar interactions strictly greater than 0. The solubility parameters δ_{d} and δₚ are defined according to the Hansen classification.

The definition and calculation of the solubility parameters in the Hansen three-dimensional solubility space are described in the paper by C. M. Hansen: “The three dimensional solubility parameters”, J. Paint Technol. 39, 105 (1967).

According to this Hansen space:

δ_{D} characterizes the London dispersion forces derived from the formation of dipoles induced during molecular impacts;
δₚ characterizes the Debye interaction forces between permanent dipoles and also the Keesom interaction forces between induced dipoles and permanent dipoles;
δₕ characterizes the specific interaction forces (such as hydrogen bonding, acid/base, donor/acceptor, etc.); and
δₐ is determined by the equation: δa=(δₚ²+δₕ¹)^{1/2}. The parameters δₚ, δₕ, δ_{d} and δₐ are expressed in (J/cm³)^{1/2}.

It may be preferable that polar oil be selected from the group consisting of plant or animal oils, such as triglycerides, ester oils, ether oils and mixtures thereof, more preferably from the group consisting of ester oils, ether oils and mixtures thereof, and even more preferably from ester oils.

The polar oil may be chosen especially from the following oils:
hydrocarbon-based polar oils such as phytostearyl esters, such as phytostearyl oleate, phytostearyl isostearate and lauroyl/octyldodecyl/phytostearyl glutamate (Ajinomoto, Eldew PS203), triglycerides consisting of fatty acid esters of glycerol, in particular the fatty acids of which may have chain lengths ranging from C₄ to C₃₆, and especially from C₁₈ to C₃₆, these oils possibly being linear or branched, and saturated or unsaturated; these oils may especially be heptanoic or octanoic triglycerides, wheatgerm oil, sunflower oil, grapeseed oil, sesame seed oil (820.6 g/mol), corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, marrow oil, blackcurrant oil, evening primrose oil, millet oil, barley oil, quinoa oil, rye oil, safflower oil, candlenut oil, passionflower oil or musk rose oil; shea butter; or alternatively caprylic/capric acid triglycerides, for instance those sold by the company Stearineries Dubois or those sold under the names Miglyol 810®, 812® and 818® by the company Dynamit Nobel;
synthetic ethers containing from 10 to 40 carbon atoms, such as dicaprylyl ether;
hydrocarbon-based esters of formula RCOOR′ in which RCOO represents a carboxylic acid residue comprising from 2 to 40 carbon atoms, and R′ represents a hydrocarbon-based chain containing from 1 to 40 carbon atoms, such as cetostearyl octanoate, isopropyl alcohol esters, such as isopropyl myristate or isopropyl palmitate, ethyl palmitate, isopropyl stearate or isostearate, isostearyl isostearate, octyl stearate, diisopropyl adipate, heptanoates, and especially isostearyl heptanoate, alcohol or polyalcohol octanoates, decanoates or ricinoleates, for instance propylene glycol dioctanoate, cetyl octanoate, tridecyl octanoate, 2-ethylhexyl 4-diheptanoate and palmitate, alkyl benzoate, polyethylene glycol diheptanoate, propylene glycol 2-diethyl hexanoate, and mixtures thereof, C₁₂ to C₁₅ alcohol benzoates, hexyl laurate, neopentanoic acid esters, for instance isodecyl neopentanoate, isotridecyl neopentanoate, isostearyl neopentanoate and 2-octyldodecyl neopentanoate, isononanoic acid esters, for instance isononyl isononanoate, isotridecyl isononanoate and octyl isononanoate, oleyl erucate, isopropyl lauroyl sarcosinate, diisopropyl sebacate, isocetyl stearate, isodecyl neopentanoate, isostearyl behenate, and myristyl myristate;
polyesters obtained by condensation of an unsaturated fatty acid dimer and/or trimer and of diol, such as those described in patent application FR 0 853 634, in particular such as dilinoleic acid and 1,4-butanediol. Mention may especially be made in this respect of the polymer sold by Biosynthis under the name Viscoplast 14436H (INCI name: dilinoleic acid/butanediol copolymer), or else copolymers of polyols and of dimer diacids, and esters thereof, such as Hailuscent ISDA;
polyol esters and pentaerythritol esters, for instance dipentaerythrityl tetrahydroxystearate/tetraisostearate;
fatty alcohols containing from 12 to 26 carbon atoms, for instance octyldodecanol, 2-butyloctanol, 2-hexyldecanol, 2-undecylpentadecanol and oleyl alcohol;
higher C₁₂-C₂₂ fatty acids, such as oleic acid, linoleic acid and linolenic acid, and mixtures thereof;
fatty acids containing from 12 to 26 carbon atoms, for instance oleic acid;
dialkyl carbonates, the two alkyl chains possibly being identical or different, such as dicaprylyl carbonate sold under the name Cetiol CC® by Cognis; and
non-volatile oils of high molecular mass, for example between 400 and 10 000 g/mol, in particular between 650 and 10 000 g/mol, for instance:
i) vinylpyrrolidone copolymers such as the vinylpyrrolidone/1-hexadecene copolymer, Antaron V-216 sold or manufactured by the company ISP (MW=7300 g/mol),
ii) esters such as:
   a) linear fatty acid esters with a total carbon number ranging from 35 to 70, for instance pentaerythrityl tetrapelargonate (MW=697.05 g/mol),
   b) hydroxylated esters such as polyglycerol-2 triisostearate (MW=965.58 g/mol),
   c) aromatic esters such as tridecyl trimellitate (MW=757.19 g/mol), C₁₂-C₁₅ alcohol benzoate, the 2-phenylethyl ester of benzoic acid, and butyloctyl salicylate,
   d) esters of C₂₄-C₂₈ branched fatty acids or fatty alcohols such as those described in patent application EP-A-0 955 039, and especially triisoarachidyl citrate (MW=1033.76 g/mol), pentaerythrityl tetraisononanoate (MW=697.05 g/mol), glyceryl triisostearate (MW=891.51 g/mol), glyceryl tris(2-decyl)tetradecanoate (MW=1143.98 g/mol), pentaerythrityl tetraisostearate (MW=1202.02 g/mol), polyglyceryl-2 tetraisostearate (MW=1232.04 g/mol) or else pentaerythrityl tetrakis(2-decyl)tetradecanoate (MW=1538.66 g/mol),
   e) esters and polyesters of dimer diol and of monocarboxylic or dicarboxylic acid, such as esters of dimer diol and of fatty acid and esters of dimer diol and of dimer dicarboxylic acid, such as Lusplan DD-DA5® and Lusplan DD-DA7® sold by the company Nippon Fine Chemical and described in patent application US 2004-175 338, the content of which is incorporated into the present application by reference,
and mixtures thereof.

The term “polar hydrocarbon-based oil” here means a polar oil formed essentially from, or even constituted by, carbon and hydrogen atoms, and optionally oxygen and nitrogen atoms, and not containing any silicon or fluorine atoms. It may contain alcohol, ester, ether, carboxylic acid, amine and/or amide groups.

It is preferable that the (c) oil has a log P value of 7.0 or less, more preferably 6.5 or less, and even more preferably 6.0 or less. It may be preferable that the (c) oil has a log P value of 1.0 or more, more preferably 1.5 or more, and even more preferably 2.0 or more. Thus, it may be preferable that the (c) oil has a log P value of from 1.0 to 7.0, more preferably from 1.5 to 6.5, and even more preferably from 2.0 to 6.0.

A log P value is a value for the base-ten logarithm of the apparent octan-1-ol/water partition coefficient. The log P values are known and are determined by a standard test which determines the concentration of the (c) oil in octan-1-ol and water. The log P may be calculated according to the method described in the article by Meylan and Howard: Atom/Fragment contribution method for estimating octanol-water partition coefficients, J. Pharm. Sci., 84: 83-92, 1995. This value may also be calculated using numerous commercially available software packages, which determine the log P as a function of the structure of a molecule. By way of example, mention may be made of the Epiwin software from the United States Environmental Agency.

The values may especially be calculated using the ACD (Advanced Chemistry Development) Solaris software V4.67; they may also be obtained from Exploring QSAR: hydrophobic, electronic and steric constants (ACS professional reference book, 1995). There is also an Internet site which provides estimated values (address: http://esc.syrres.com/interkow/kowdemo.htm).

The (c) oil may have at least two moieties selected from the group consisting of an amide bond, an ester bond, and mixtures thereof. The amide bond here means —CONR— (R denotes a hydrogen atom or a linear or branched C₁-C₁₈ alkyl group, preferably a methyl group) and the ester bond here means —COO—. In other words, the (c) oil may have two or more amide bonds, two or more ester bonds or a mixture of at least one amide bond and at least one ester bond.

The (c) oil may have at least two moieties selected from the group consisting of an ether bond, an ester bond, and mixtures thereof. The ether bond here means —O— and the ester bond here means —COO—. In other words, the (c) oil may have two or more ether bonds, two or more ester bonds or a mixture of at least one ether bond and at least one ester bond.

It is preferable that the (c) oil be selected from the group consisting of isopropyl lauroyl sarcosinate, bis-ethoxydiglycol cyclohexane 1,4-dicarboxylate and mixtures thereof.

The amount of the (c) oil(s) in the composition according to the present invention may be 0.01% by weight or more, preferably 0.1% by weight or more, and more preferably 1% by weight or more, relative to the total weight of the composition. It may be even more preferable that the amount of the (c) oil(s) in the composition according to the present invention be 3% by weight or more, relative to the total weight of the composition.

On the other hand, the amount of the (c) oil(s) in the composition according to the present invention may be 50% by weight or less, preferably 30% by weight or less, and more preferably 15% by weight or less, relative to the total weight of the composition. It may be even more preferable that the amount of the (c) oil(s) in the composition according to the present invention be 10% by weight or less, relative to the total weight of the composition.

The amount of the (c) oil(s) in the composition according to the present invention may range from 0.01% to 50% by weight, preferably from 0.1% to 30% by weight, more preferably from 1% to 15% by weight, relative to the total weight of the composition. It may be even more preferable that the amount of the (c) oil(s) in the composition according to the present invention be from 3% to 10% by weight, relative to the total weight of the composition.

It may be preferable that the weight ratio of the (a) at least one cinnamic acid derivative represented by the above chemical formula (I)/the (c) at least one oil in the composition according to the present invention be less than 0.1, more preferably less than 0.09, and more preferably less than 0.85. If this weight ratio is 0.1 or more, the stability of the composition may be deteriorated because crystallization may occur at a relatively low temperature.

The (c) oil(s) can form a fatty phase of the composition according to the present invention.

If the composition according to the present invention is in the form of the O/W type, the (c) oil in the composition according to the present invention can form dispersed fatty phases in the O/W type composition.

[Water]

The composition according to the present invention comprises (d) water.

The (d) water can form an aqueous phase of the composition according to the present invention.

If the composition according to the present invention is in the form of the O/W type, the (d) water in the composition according to the present invention can form a continuous aqueous phase in the O/W type composition.

The amount of the (d) water in the composition according to the present invention may be 50% by weight or more, preferably 60% by weight or more, and more preferably 70% by weight or more, relative to the total weight of the composition.

On the other hand, the amount of the (d) water in the composition according to the present invention may be 90% by weight or less, preferably 85% by weight or less, and more preferably 80% by weight or less, relative to the total weight of the composition.

The amount of (d) water in the composition according to the present invention may range from 50% to 90% by weight, preferably from 60% to 85% by weight, more preferably from 70% to 80% by weight, relative to the total weight of the composition.

[Polysaccharide]

The composition according to the present invention may comprise (e) at least one polysaccharide. A single type of polysaccharide may be used, but two or more different types of polysaccharide may be used in combination.

The (e) at least one polysaccharide can form a capsule which can encapsulate the (a) at least one cinnamic acid derivative according to the above chemical formula (I), preferably together with the (c) at least one oil, and more preferably together with the (b) at least one lipophilic antioxidant and the (c) at least one oil. The encapsulation of the (a) at least one cinnamic acid derivative according to the above chemical formula (I) can further reduce yellowing or bad odor of the composition according to the present invention. Thus, the capsule can further enhance the stability of the composition according to the present invention.

The capsule of the (e) at least one polysaccharide including the (a) at least one cinnamic acid derivative according to the above chemical formula (I) can be prepared by surrounding or coating the (a) at least one cinnamic acid derivative according to the above chemical formula (I) with at least one layer or film of the (e) at least one polysaccharide. It is preferable that a mixture of the (a) at least one cinnamic acid derivative according to the above chemical formula (I) and the (c) at least one oil be surrounded or coated by the (e) at least one polysaccharide. It is more preferable that a mixture of the (a) at least one cinnamic acid derivative according to the above chemical formula (I), the (b) at least one lipophilic antioxidant and the (c) at least one oil be surrounded or coated by the (e) at least one polysaccharide. It is even more preferable that the (a) at least one cinnamic acid derivative according to the above chemical formula (I) be solubilized into the (c) at least one oil, and then the (c) at least one oil be surrounded or coated by the (e) at least one polysaccharide. The (b) at least one lipophilic antioxidant may also be solubilized into the (c) at least one oil.

The surrounding step may be performed by any conventional process. For example, it is possible to coextrude (i) a mixture of the (a) at least one cinnamic acid derivative according to the above chemical formula (I) and the (c) at least one oil together with (ii) the (e) polysaccharide. In this, case, the excluded (i) mixture can form a core, while the (ii) (e) polysaccharide can form a shell. The coextruded core/shell structure can transform into a core/shell particle which corresponds to the capsule. The above (i) mixture may also include the (b) at least one lipophilic antioxidant.

The form of the capsule is not limited. For example, the capsule may be in the form of a sphere.

The size of the capsule is not limited. It is possible that the size or the diameter of the capsule may be from 0.1 to 10 mm, preferably from 0.5 to 5 mm, and more preferably from 1 to 3 mm.

The capsule can be dispersed and suspended in the composition according to the present invention. The capsule can provide the composition according to the present invention with unique aspects.

It is preferable that the (e) polysaccharide be selected from polysaccharides derived from plants. In other words, it is preferable that the (e) polysaccharide be of plant origin.

On the other hand, it is also preferable that the (e) polysaccharide not be selected from cellulose and derivatives thereof.

According to the present invention, the term “polysaccharides derived from plants” especially means polysaccharides obtained from the plant kingdom (plants or algae), as opposed to polysaccharides obtained via biotechnology, as is the case, for example, for xanthan gum, which is produced especially by fermentation of a bacterium, Xanthomonas campestris.

As examples of polysaccharides of plant origin that may be used according to the present invention, mention may be made especially of:
a) algal extracts, such as alginates, carrageenans and agars, and mixtures thereof. Examples of carrageenans that may be mentioned include Satiagum UTC30® and UTC10® from the company Degussa; an alginate that may be mentioned is the sodium alginate sold under the name Kelcosol® by the company ISP;
b) gums, such as guar gum and nonionic derivatives thereof (hydroxypropyl guar), gum arabic, konjac gum or mannan gum, gum tragacanth, ghatti gum, karaya gum or locust bean gum; examples that may be mentioned include the guar gum sold under the name Jaguar HP105® by the company Rhodia; the mannan and konjac Gum® (1% gluconomannan) sold by the company GfN;
c) modified or unmodified starches, such as those obtained, for example, from cereals, for instance wheat, corn or rice, from legumes, for instance blonde pea, from tubers, for instance potato or cassava, and tapioca starches; dextrins, such as corn dextrins; examples that may especially be mentioned include the rice starch Remy DR I® sold by the company Remy; the corn starch B® from the company Roquette; the potato starch modified with 2-chloroethylaminodipropionic acid neutralized with sodium hydroxide, sold under the name Structure Solanace® by the company National Starch; the native tapioca starch powder sold under the name Tapioca Pure® by the company National Starch;
d) dextrins, such as the dextrin extracted from corn under the name Index® from the company National Starch; and
   mixtures thereof.

Preferably, the (e) polysaccharide be chosen from algal extracts.

The algal extracts may be chosen from alginates, carrageenans and agars, and mixtures thereof. Preferably, alginates or agars, or mixtures thereof, may be used.

The amount of the (e) polysaccharide(s) in the composition according to the present invention may be 0.001% by weight or more, preferably 0.005% by weight or more, and more preferably 0.01% by weight or more, relative to the total weight of the composition. It may be even more preferable that the amount of the (e) polysaccharide(s) in the composition according to the present invention be 0.03% by weight or more, relative to the total weight of the composition.

On the other hand, the amount of the (e) polysaccharide(s) in the composition according to the present invention may be 5% by weight or less, preferably 2% by weight or less, and more preferably 1% by weight or less, relative to the total weight of the composition. It may be even more preferable that the amount of the (e) polysaccharide(s) in the composition according to the present invention be 0.1% by weight or less, relative to the total weight of the composition.

The amount of the (e) polysaccharide(s) in the composition according to the present invention may range from 0.001% to 5% by weight, preferably from 0.005% to 2% by weight, more preferably from 0.01% to 1% by weight, relative to the total weight of the composition. It may be even more preferable that the amount of the (e) polysaccharide(s) in the composition according to the present invention be from 0.03% to 0.1% by weight, relative to the total weight of the composition.

[pH]

The pH of the composition according to the present invention is 4.5 or less, preferably 4.4 or less, and more preferably 4.3 or less. The pH of the composition according to the present invention may be 3.0 to 4.5, preferably from 3.5 to 4.5, and more preferably from 4.0 to 4.5.

At a pH of 4.5 or less, the composition according to the present invention is stable.

The pH of the composition means the pH of the aqueous phase of the composition according to the present invention. In particular, pH of the composition means the pH of the (d) water in the composition according to the present invention.

The pH of the composition according to the present invention may be adjusted by adding at least one acidifying agent and/or at least one basifying agent (alkaline agent).

The acidifying agents can be, for example, mineral or organic acids, for instance hydrochloric acid, phosphoric acid, carboxylic acids, for instance tartaric acid, citric acid, and lactic acid, or sulphonic acids.

The acidifying agent may be present in an amount ranging from less than 5% by weight, preferably from 3% by weight or less, and more preferably from 1% by weight or less, relative to the total weight of the composition.

The basifying agent or alkaline agent can be, for example, any inorganic or organic basic agents which are commonly used in cosmetic products such as ammonia; alkanolamines such as mono-, di- and tri-ethanolamine, isopropanolamine; metal hydroxide such as alkaline metal hydroxide (e.g., sodium and potassium hydroxides); urea, guanidine and their derivatives; and diamines such as those described in the structure below:

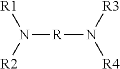

wherein
R denotes an alkylene such as propylene optionally substituted by a hydroxyl or a C₁-C₄ alkyl radical, and R₁, R₂, R₃, and R₄ independently denote a hydrogen atom, an alkyl radical, or a C₁-C₄ hydroxyalkyl radical, which may be exemplified by 1,3-propanediamine, and derivatives thereof. Alkaline metal hydroxide such as sodium hydroxide may be preferable.

The composition according to the present invention may comprise at least one water-miscible solvent such as a lower monoalcohol containing from 1 to 5 carbon atoms, C₃-C₄ ketones or C₃-C₄ aldehydes. The water-miscible solvent that can preferably be used is ethanol. The content of water-miscible solvent can range from 0.1% to 15% by weight, and better still from 1% to 8% by weight, relative to the total weight of the composition.

It is a matter of routine operation for a person skilled in the art to adjust the nature and amount of the above optional additives which may be present in the composition in accordance with the present invention such that the desired cosmetic properties are not thereby affected.

[Polyol]

The composition according to the present invention may further comprise at least one polyol. A single type of polyol may be used, but two or more different types of polyol may be used in combination.

The term “polyol” here means an alcohol having two or more hydroxy groups, and does not encompass a saccharide or a derivative thereof. The derivative of a saccharide includes a sugar alcohol which is obtained by reducing one or more carbonyl groups of a saccharide, as well as a saccharide or a sugar alcohol in which the hydrogen atom or atoms in one or more hydroxy groups thereof has or have been replaced with at least one substituent such as an alkyl group, a hydroxyalkyl group, an alkoxy group, an acyl group or a carbonyl group.

The polyol may be a C₂-C₁₂ polyol, preferably a C₂-C₉ polyol, comprising at least 2 hydroxy groups, and preferably 2 to 5 hydroxy groups.

The polyol may be a natural or synthetic polyol. The polyol may have a linear, branched or cyclic molecular structure.

The polyol may be selected from glycerins and derivatives thereof, and glycols and derivatives thereof. The polyol may be selected from the group consisting of glycerin, diglycerin, polyglycerin, ethyleneglycol, diethyleneglycol, propyleneglycol, dipropyleneglycol, butyleneglycol, pentyleneglycol, hexyleneglycol, 1,3-propanediol, 1,5-pentanediol, polyethyleneglycol (5 to 50 ethyleneoxide groups), and sugars such as sorbitol.

The amount of the polyol(s) in the composition according to the present invention may be 0.01% by weight or more, preferably 0.05% by weight or more, and more preferably 0.1% by weight or more, relative to the total weight of the composition.

On the other hand, the amount of the polyol(s) in the composition according to the present invention may be 30% by weight or less, preferably 20% by weight or less, and more preferably 15% by weight or less, relative to the total weight of the composition.

Thus, the polyol(s) may be present in the composition according to the present invention in an amount ranging from 0.01% to 30% by weight, and preferably from 0.05% to 20% by weight, such as from 0.1% to 15% by weight, relative to the total weight of the composition.

[Other Ingredients]

The composition according to the present invention may contain one or more monoalcohols which are in the form of a liquid at room temperature (25° C.), such as for example linear or branched monoalcohols comprising from 1 to 6 carbon atoms, such as ethanol, propanol, butanol, isopropanol, isobutanol, pentanol, and hexanol.

The amount of the monoalcohol(s) in the composition according to the present invention may be 0.01% by weight or more, preferably 0.1% by weight or more, and more preferably 1% by weight or more, relative to the total weight of the composition.

On the other hand, the amount of the monoalcohol(s) in the composition according to the present invention may be 15% by weight or less, preferably 10% by weight or less, and more preferably 5% by weight or less, relative to the total weight of the composition.

Thus, the amount of the monoalcohol(s) in the composition according to the present invention may range from 0.01% to 15% by weight, preferably from 0.1% to 10% by weight, and more preferably from 1% to 5% by weight, relative to the total weight of the composition.

The composition according to the present invention may also include various adjuvants conventionally used in cosmetic and dermatological compositions, such as anionic, non-ionic, cationic, and amphoteric or zwitterionic polymers, anionic, cationic, amphoteric, and nonionic surfactants, hydrophilic antioxidants, coloring agents, chelating agents, sequestering agents, fragrances, dispersing agents, conditioning agents, film-forming agents, preservatives, co-preservatives, and mixtures thereof, except for the ingredients as explained above.

[Preparation]

The composition according to the present invention can be prepared by mixing the essential ingredient(s) as explained above, and optional ingredient(s), if necessary, as explained above.

The method and means to mix the above essential and optional ingredients are not limited. Any conventional method and means can be used to mix the above essential and optional ingredients to prepare the composition according to the present invention.

[Form]

The form of the composition according to the present invention is not particularly limited, and may take various forms such as an emulsion (O/W or W/O form), an aqueous gel, an aqueous solution, or the like.

It is preferable that the composition according to the present invention be in the form of O/W type. It is more preferable that the composition according to the present invention be in the form of an O/W emulsion or an O/W dispersion which comprises fatty phases dispersed in a continuous aqueous phase. The dispersed fatty phases can be oil droplets in the aqueous phase. It is even more preferable that the composition according to the present invention be in the form of an O/W gel emulsion or an O/W gel dispersion.

The O/W architecture or structure, which consists of fatty phases dispersed in an aqueous phase, has an external aqueous phase, and therefore if the composition according to the present invention has the O/W architecture or structure, it can provide a pleasant feeling during use because of the feeling of immediate freshness that the aqueous phase can provide.

[Process and Use]

It is preferable that the composition according to the present invention be a cosmetic or dermatological composition, preferably a cosmetic composition, and more preferably a cosmetic composition for a keratin substance such as skin.

The composition according to the present invention can be used for a non-therapeutic process, such as a cosmetic process, for treating a keratin substance such as skin, hair, mucous membranes, nails, eyelashes, eyebrows and/or scalp, by being applied to the keratin substance.

Thus, the present invention also relates to a cosmetic process for treating a keratin substance such as skin, comprising the step of applying the composition according to the present invention to the keratin substance.

The composition according to the present invention may be used as an anti-oxidant, whitening, or UV filtering product for a keratinous substance such as skin. In particular, the composition according to the present invention may be used as a skin whitening cosmetic product.

Another aspect of the present invention relates to a use of:

(b) at least one lipophilic antioxidant;
(c) at least one oil
for the manufacture of a stable composition with a pH of 4.5 or less comprising (a) at least one cinnamic acid derivative represented by the above chemical formula (I) and (d) water.

Another aspect of the present invention also relates to a process for preparing a stable composition comprising

(a) at least one cinnamic acid derivative represented by the above chemical formula (I), and (d) water,
comprising a step of adding
(b) at least one lipophilic antioxidant; and
(c) at least one oil
to the composition with a pH of 4.5 or less.

In the above aspects, it is preferable that the (a) at least one cinnamic acid derivative represented by the above chemical formula (I) be encapsulated by the (e) at least one polysaccharide.

It is more preferable that a mixture of the (a) at least one cinnamic acid derivative represented by the above chemical formula (I) and the (c) at least one oil be encapsulated by the (e) at least one polysaccharide.

It is even more preferable that a mixture of the (a) at least one cinnamic acid derivative represented by the above chemical formula (I), the (b) at least one lipophilic antioxidant, and the (c) at least one oil be encapsulated by the (e) at least one polysaccharide.

The above explanations regarding the ingredients (a) to (e), as well as the optional ingredients, for the composition according to the present invention can apply to those for the above uses and processes according to the present invention. The explanations regarding the preparation and forms of the composition according to the present invention can also apply to those of the composition recited in the above uses and processes.

### EXAMPLES

The present invention will be described in more detail by way of examples which however should not be construed as limiting the scope of the present invention.

### Examples 1-4 and Comparative Examples 1-6

The following compositions according to Examples 1-4 and Comparative Examples 1-6, shown in Table 1, were prepared by mixing the ingredients shown in Table 1.

The preparations of the O/W emulsions (Examples 1-2 and Comparative Examples 1-4) were performed as follows:

(1) mixing the ingredients in Phases A and B with a homogenizer at 75-80° C.; and
(2) cooling the mixture obtained in the above step (1) to a room temperature.

The preparations of the encapsulated O/W gel type dispersions (Examples 3-4 and Comparative Examples 5-6) were performed as follows:
(1) mixing the ingredients of Phase A at 75-80° C. to form a uniform mixture of Phase A;
(2) mixing the ingredients of Phase B at 75-80° C. to form a uniform mixture of Phase B;
(3) mixing the ingredients of Phase C at 80-90° C. to form a uniform mixture of Phase C;
(4) coextruding the uniform mixture of Phase A and the uniform mixture of Phase C by encapsulating the former with the latter to prepare capsules in which the uniform mixture of Phase A is coated and encapsulated with the uniform mixture of Phase C;
(5) adding the capsules to the uniform mixture of Phase B; and
(6) cooling the mixture obtained in the above step (5) to a room temperature.

The numerical values for the amounts of the components shown in Table 1 are all based on “% by weight” as active raw materials.

TABLE 1

| | | | | | | | | | | Encapsulated O/W | |
|---|---|---|---|---|---|---|---|---|---|---|---|
| | | | | Encapsulated O/W | | O/W Emulsion | | | | Gel Type Dispersion | |
| | | O/W Emulsion | | Gel Type Dispersion | | Comp. | Comp. | Comp. | Comp. | Comp. | Comp. |
| | Ingredients | Ex. 1 | Ex. 2 | Ex. 3 | Ex. 4 | Ex. 1 | Ex. 2 | Ex. 3 | Ex. 4 | Ex. 5 | Ex. 6 |
| A | Ferulic | 0.50 | 0.50 | 0.50 | 0.50 | 0.5 | 0.5 | 0.5 | 0.50 | 0.50 | 0.50 |
| | Acid | | | | | | | | | | |
| | Capryloyl | — | — | 0.15 | 0.15 | — | — | — | — | 0.15 | 0.15 |
| | Salicylic Acid | | | | | | | | | | |
| | Phenylethyl | — | — | 0.30 | 0.30 | — | — | — | — | 0.30 | 0.30 |
| | Resorcinol | | | | | | | | | | |
| | Pentaerythrityl | 0.05 | — | 0.025 | 0.025 | 0.05 | 0.05 | — | 0.05 | 0.025 | — |
| | Tetra-di-t-butyl | | | | | | | | | | |
| | hydroxyhydrocinnamate | | | | | | | | | | |
| | Tocophenol | — | 0.10 | 0.015 | 0.015 | — | — | — | — | 0.015 | — |
| | Isopropyl | 6.00 | 6.00 | 6.00 | 6.00 | 6.00 | 6.00 | 6.00 | — | 6.00 | 6.00 |
| | Lauroyl Sarcosinate | | | | | | | | | | |
| | Caprylic/ | 0.50 | 0.50 | 0.50 | 0.50 | 0.50 | 0.50 | 0.50 | 6.50 | 0.50 | 0.50 |
| | Capric/Succinic | | | | | | | | | | |
| | Triglyceride | | | | | | | | | | |
| | Fragrance | 0.15 | 0.15 | 0.15 | 0.15 | 0.15 | 0.15 | 0.15 | 0.15 | 0.15 | 0.15 |
| B | Trisodium | 0.25 | 0.25 | 0.25 | 0.25 | 0.25 | 0.25 | 0.25 | 0.25 | 0.25 | 0.25 |
| | Ethylenediamine | | | | | | | | | | |
| | Disuccinate | | | | | | | | | | |
| | Acrylates/C10-30 | 0.40 | 0.40 | — | — | 0.40 | 0.40 | 0.40 | 0.40 | — | — |
| | Alkyl Acrylate | | | | | | | | | | |
| | Crosspolymer | | | | | | | | | | |
| | Xanthan Gum (and) | 0.12 | 0.12 | 0.12 | 0.12 | 0.12 | 0.12 | 0.12 | 0.12 | 0.12 | 0.12 |
| | Ceratonia Siliqua | | | | | | | | | | |
| | (Carob) Gum | | | | | | | | | | |
| | Sodium | 0.10 | 0.10 | 0.10 | 0.10 | 0.10 | 0.10 | 0.10 | 0.10 | 0.10 | 0.10 |
| | Hyaluronate | | | | | | | | | | |
| | Cellulose Gum | — | — | 0.60 | 0.60 | — | — | — | — | 0.60 | 0.60 |
| C | Agar | — | — | 0.035 | 0.035 | — | — | — | — | 0.035 | 0.035 |
| | Arginate | — | — | 0.017 | 0.017 | — | — | — | — | 0.017 | 0.017 |
| | Water | — | — | 3.20 | 3.20 | — | — | — | — | 3.20 | 3.20 |
| B | Chlorphenes in | 0.25 | 0.25 | 0.25 | 0.25 | 0.25 | 0.25 | 0.25 | 0.25 | 0.25 | 0.25 |
| | Hydroxyacetophenone | 0.50 | 0.50 | 0.50 | 0.50 | 0.50 | 0.50 | 0.50 | 0.50 | 0.50 | 0.50 |
| | Caprylyl | 0.20 | 0.20 | 0.20 | 0.20 | 0.20 | 0.20 | 0.20 | 0.20 | 0.20 | 0.20 |
| | Glycol | | | | | | | | | | |
| | Dipropylene | 2.00 | 2.00 | 2.00 | 2.00 | 2.00 | 2.00 | 2.00 | 2.00 | 2.00 | 2.00 |
| | Glycol | | | | | | | | | | |
| | Butylene | 10.00 | 10.00 | 10.00 | 10.00 | 10.00 | 10.00 | 10.00 | 10.00 | 10.00 | 10.00 |
| | Glycol | | | | | | | | | | |
| | Glycerin | 3.00 | 3.00 | 3.00 | 3.00 | 3.00 | 3.00 | 3.00 | 3.00 | 3.00 | 3.00 |
| | Citric | qs | qs | qs | qs | qs | qs | qs | qs | qs | qs |
| | Acid | pH 4.0 | pH 4.0 | pH 4.0 | pH 4.5 | pH 5.0 | pH 5.5 | pH 4.0 | pH 4.0 | pH 5.0 | pH 4.0 |
| | Water | qsp | qsp | qsp | qsp | qsp | qsp | qsp | qsp | qsp | qsp |
| | | 100 | 100 | 100 | 100 | 100 | 100 | 100 | 100 | 100 | 100 |
| Weight | Ferulic Acid/Isopropyl | 0.083 | 0.083 | 0.083 | 0.083 | 0.083 | 0.083 | 0.083 | — | 0.083 | 0.083 |
| Ratio | Lauroyl Sarcosinate | | | | | | | | | | |
| Stability | Yellowing | Good | Good | Very | Good | Poor | Very | Poor | Good | Poor | Poor |
| | | | | Good | | | Poor | | | | |
| | Crystallization | Very | Very | Very | Very | Very | Very | Very | Very | Very | Very |
| | | Good | Good | Good | Good | Good | Good | Good | Poor | Good | Good |
| | Odor | Good | Good | Very | Good | Poor | Very | Poor | Good | Poor | Poor |
| | | | | Good | | | Poor | | | | |

[Evaluations]

(Stability)

Each of the compositions according to Examples 1-4 and Comparative Examples 1-6 was filled into four glass bottles, and each of the glass bottles was held under temperature conditions of 4° C., 25° C., 40° C., and 45° C., respectively, for 2 months.

Each glass bottle was then investigated for the degree of change (in terms of color, crystallization and odor), and evaluated by the following criteria.
Very Good: Almost the same condition as production.
Good: Little change in color, odor, or aspect was observed.
Poor: Change in color, odor, or aspect could be clearly observed. Yellowing, crystallization or bad odor could be clearly observed.
Very Poor: Change in color, odor, or aspect could be remarkably noticed. Yellowing, crystallization or bad odor could be remarkably noticed.

The results are shown in Table 1.

It should be noted that Examples 1-2 should be compared with Comparative Examples 1-4, and that Examples 3-4 should be compared with Comparative Examples 5-6.

The compositions according to Examples 1-2 and Comparative Examples 1-4 were in the form of an O/W emulsion.

The compositions according to Examples 1-2 which comprise all the ingredients (a) to (d) explained above was stable.

The compositions according to Comparative Examples 1-2 which had a pH of more than 4.5 (5.0 and 5.5, respectively) were not stable.

The compositions according to Comparative Examples 3-4 which lack the (c) lipophilic antioxidant were not stable.

The compositions according to Examples 3-4 and Comparative Examples 5-6 were in the form of an encapsulated oil-in-water gel type dispersion, in which the (a) oil is encapsulated in capsules formed by the (e) polysaccharide. A plurality of the capsules are present in the compositions according to Examples 3-4 and Comparative Examples 5-6.

The compositions according to Examples 5-6 which comprise all the ingredients (a) to (e) explained above was stable.

The composition according to Comparative Example 5 which had a pH of more than 4.5 (5.0) was not stable.

The composition according to Comparative Example 6 which lacked the (c) lipophilic antioxidant was not stable.

1. A composition comprising:
(a) at least one cinnamic acid derivative represented by the following chemical formula (I) 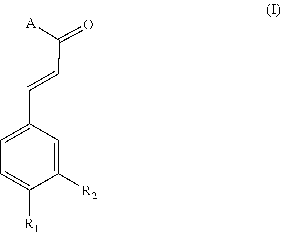
wherein
A is chosen from
an OR₃ group wherein R₃ is chosen from a hydrogen atom, a phytyl group, a benzyl group, a linear or branched C₁-C₁₈ alkyl group, a C₃-C₈ cycloalkyl group, a C₃-C₈ cycloalkyl-C₁-C₅ alkyl group, an alkali metal ion, an alkaline earth metal ion and an ammonium ion,
and
an NHR₄ group wherein R₄ is chosen from a hydrogen atom, a phytyl group, a benzyl group, and a linear or branched C₁-C₁₈ alkyl group, a C₃-C₈ cycloalkyl group, a C₃-C₈ cycloalkyl-C₁-C₅ alkyl group,
R₁ is chosen from a hydrogen atom, a hydroxyl group, a C₁-C₆ alkoxy group, a linear or branched C₁-C₁₈ alkyl group, a C₃-C₈ cycloalkyl group, a C₃-C₈ cycloalkyl-C₁-C₅ alkyl group, and
R₂ is chosen from a hydrogen atom, a hydroxyl group, and a C₁-C₆ alkoxy group;
(b) at least one lipophilic antioxidant;
(c) at least one oil; and
(d) water,
wherein
the pH of the composition is 4.5 or less.

2. The composition according to claim 1, wherein the (a) cinnamic acid derivative is ferulic acid.

3. The composition according to claim 1, wherein the amount of the (a) cinnamic acid derivative(s) in the composition ranges from 0.01% to 8% by weight relative to the total weight of the composition.

4. The composition according to claim 1, wherein the (b) lipophilic antioxidant is selected from tocopherol, pentaerythrityl tetra-di-t-butyl hydroxyhydrocinnamate, and a mixture thereof.

5. The composition according to claim 1, wherein the amount of the (b) lipophilic antioxidant(s) in the composition ranges from 0.001% to 10% by weight relative to the total weight of the composition.

6. The composition according to claim 1, wherein the (a) cinnamic acid derivative is soluble in the (c) oil.

7. The composition according to claim 1, wherein the (c) oil is selected from the group consisting of isopropyl lauroyl sarcosinate, bis-ethoxydiglycol cyclohexane 1,4-dicarboxylate, and mixtures thereof.

8. The composition according to claim 1, wherein the amount of the (c) oil(s) in the composition ranges from 0.01% to 50% by weight relative to the total weight of the composition.

9. The composition according to claim 1, wherein the amount of the (d) water in the composition ranges from 50% to 90% by weight relative to the total weight of the composition.

10. The composition according to claim 1, wherein the composition further comprises (e) at least one polysaccharide.

11. The composition according to claim 10, wherein the (e) polysaccharide is selected from agar, alginate, carrageenan and a mixture thereof.

12. The composition according to claim 10, wherein the amount of the (e) polysaccharide(s) in the composition ranges from 0.001% to 5% by weight relative to the total weight of the composition.

13. The composition according to claim 1, wherein the composition is in the O/W form.

14. The composition according to claim 1, wherein the composition is a cosmetic composition.

15. A cosmetic process for treating a keratin substance, comprising the step of applying the composition according to claim 1 to the keratin substance.
